(19) United States
(12) Patent Application Publication
(10) Pub. No.: US 2002/0081627 A1
(43) Pub. Date: Jun. 27, 2002

(54) Inhibition of adenovirus replication by product R, A peptide nucleic acid immunomodulator

(76) Inventor: Shalom Z. Hirschman, Riverdale, NY (US)

Correspondence Address: COHEN, PONTANI, LIEBERMAN & PAVANE, Suite 1210, 551 Fifth Avenue, New York, NY, 10176, US

(21) Appl. No.: 09/948,221

(22) Filed: Sep. 7, 2001

Related U.S. Application Data
(60) Provisional application No. 60/231,073, filed on Sep. 8, 2000.

(51) Int. Cl. C12Q 1/70; A61K 38/16; A01N 37/18; A61K 38/00; A61K 39/23; A61K 39/235; G01N 33/53

(52) U.S. Cl. 435/7.1; 514/2; 435/5; 424/233.1

(57) ABSTRACT

The invention relates to a method of inhibiting adenovirus replication. The method comprises the step of contacting Product R with cells that are infected with adenovirus. The invention provides a basis for the treatment of adenovirus infections in human such as pink eye disease.

### RELATED APPLICATIONS

This application claims priority from U.S. Provisional Patent Application Serial No. 60/231,073 which was filed on Sep. 08, 2000, the content of which is hereby incorporated by reference in its entirety.

### BACKGROUND OF THE INVENTION

1. Field of the Invention

The present invention relates to a method of inhibiting adenovirus replication by contacting Product R, a pepetide nucleic acid immunonodulator with host cells that are infected by adenovirus.

2. Description of the Related Art

Adenovirus is an ideal study model for the interaction between host cells and infecting viruses at the gene regulation system (reference 7). E2F was identified originally by its DNA-binding activity with specific recognition sequences in adenovirus E2 promoter. The viral E1A protein can induce E2F mediated DNA binding and transcriptional activities by releasing free E2F from inactive protein complex (references 1, 2, 4, 8 and 9). The significance of these findings was limited until the discoveries that promoters of many cellular genes contain similar E2F-binding sites and that E2F is one of the important cellular transcription factors in regulating expression of some of these genes. Many of these genes are involved in cell cycle progression, particularly in DNA synthesis. Furthermore, several key regulators of the cell cycle, such as retinoblastoma protein (Rb) and related proteins p107 and p130, were found to form complexes with E2F, indicating the potential role of E2F in cell cycle progression. By its ability to bind to the Rb protein, E2F-1 was the first gene product identified amongst a family of E2F transcription factors (reference 5,). As an authentic transcription factor, E2F-1 contains a specific DNA-binding domain and a potent transactivation region. E2F-1 can form heterodimers with another E2F-like protein, DP-1, and have a synergistic effect on its transcription activity. The Rb-binding domain of E2F-1 overlaps its transcriptional activation region, suggesting a possible mechanism for Rb to regulate E2F-1 transcriptional activity. Indeed, it has been shown that Rb suppresses transcriptional activation mediated by E2F-1 through the direct interaction between the two molecules. The inhibitory effects of Rb can be disrupted by viral oncoproteins, such as E1A, which can directly interact with the Rb protein (reference 6). Similar inhibition can also be achieved by inactivation of Rb protein through mutations and phosphorylation. A noteworthy observation made during the original characterization of E2F-1 was that E2f -1 expression is cell cycle dependent, with a peak at the G₁/S boundary. This finding is consistent with the hypothesis that E2F functions primarily at this period of time in the cell cycle and E2F-1 mediated transcriptional activation may be one of the rate-limiting steps in cell proliferation (references 3, 4 and 10). The inducible expression of an exogenous E2F-1 gene was employed to examine the consequences of the deregulated expression of E2F-1in Rat-2 fibroblasts which induces premature S-phase entry and subsequently leads to apoptotic cell death.

Product R is peptide nucleic acid preparation with antiviral and immunnomodulating properties. In addition to its antiviral effects through the immune system of human body, the previous studies have shown that Product R exhibits direct effects of anti-human immunodeficiency virus and anti-human papilloma virus in cell culture systems (references 11, 12 and 13).

However, there is no teaching or suggestion in prior art that Product R could directly act on adenovirus to inhibit adenovirus replication without the presence of the functions of the immune system.

The following references provide more comprehensive descriptions of the state of art. These references are hereby incorporated by reference in their entireties.

References:

1. Dyson, N. 1998 . The regulation of E2F by Rb-family proteins. Genes Dev. 2:2245-2262

2. Levine, A. J. 1997. P53, the cellular gatekeeper or growth and division. Cell 88:323-331

3. Moran, E. 1993 . DNA tumor viruses and the cell cycle. Curr. Opin. Genet. Dev. 3:63-70

4. Nevins, J. R. 1992. E2F: a link between the Rb tumor suppressor protein and viral oncoproteins. Science 58:424-429

5. Weinberg, R. A. 1995. The retinoblastoma protein and cell cycle control. Cell 81:323- 330

6. Egan, C., S. T. Bayler, and P. E. Branton. 1989. Binding of the Rb1 protein to E1A products is required for adenovirus transformation. Oncogene 4:383-388

7. Moore, M., N. Horikoshi, and T. Shenk. 1996. Oncogenic potential of the adenovirus Orf6 protein. Proc. Natl. Acad. Sci. USA 93:11295-11301

8. Somasundaram, K., and W. S. EI-Deiry. 1997. Inhibition of P53-mediated transactivation and cell-cycle arrest by E1A through its p300/CBP-interacting region. Oncogene 14:1047-1057

9. Shenk. T, Flint J. 1991. Transcriptional and transforming activities of the adenovirus E1A proteins. Adv. Cancer Res. 57:47-85

10. Slomiany B A, D'Arigo K L, Kelly M M, Kurtz D T. 2000. C/EBP alpha inhibits cell growth via direct repression of E2F-DP mediated transcription. Mol Cell Biol 20(16):5986-973.

11. Hirschman S Z and Chen C W. 1996. Peptide nucleic acids stimulate gamma interferon and inhibit the replication of the Human Immunodeficiency Virus. J. Invest. Med. 44:347-351

12. Deboral A. Lazzarino, Maribel de Diego, Elgilda Musi, Shalom Z. Hirschman, and Richard J. Alexander CXCR4 and CCR5 Expression by H9 T-cells is down-regulated by a peptide-nucleic acid immunomodulator. Immunology Letters (in press).

13. Wenlin Huang, Jin Zhang, Yuanen Ji, John Fisher, Shalom Z. Hirschman. 2000, Product R, regulates human papilloma virus-18 E7 oncogene expression in Hela cells. Journal of Infectious Diseases, 31-1, 320-221. 2000.

### SUMMARY OF THE INVENTION

It is an object of the present invention to establish a method of inhibiting adenovirus replication by directly contacting Product R with host cells infected by adenovirus.

Another object of the present invention is to establish a method of reducing adenovirus induced apoptosis by modulating the cell cycles.

A further object of the present invention is to establish a method demonstrating the ability of Product R in altering steady state levels of adenovirus EIA protein.

Adenovirus infections cause epidemic cojunctivitis (“pink eye”), upper and lower respiratory illness and gastronenteritis. There currently is no approved and non-toxic treatment for adenoviral infections. The present invention provides a scientific rationale for an effort to develop an ophthalmic formulation of Product R for potential treatment of “pink eye”. In addition, the present invention hold promise for a large number of patients infected by adenoviruses every year and who are afflicted with respiratory and gastrointestinal illnesses. Moreover, present invention provide hope for patients with compromised immune systems who may suffer with more severe adenoviral infections such as diffuse pneumonias.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims. It should be further understood that the drawings are not necessarily drawn to scale and that, unless otherwise indicated, they are merely intended to conceptually illustrate the structures and procedures described herein.

### BRIEF DESCRIPTION OF THE DRAWINGS

In the drawings:

FIG. 1. Schematic of adenovirus type 5 Lac Z marker gene.

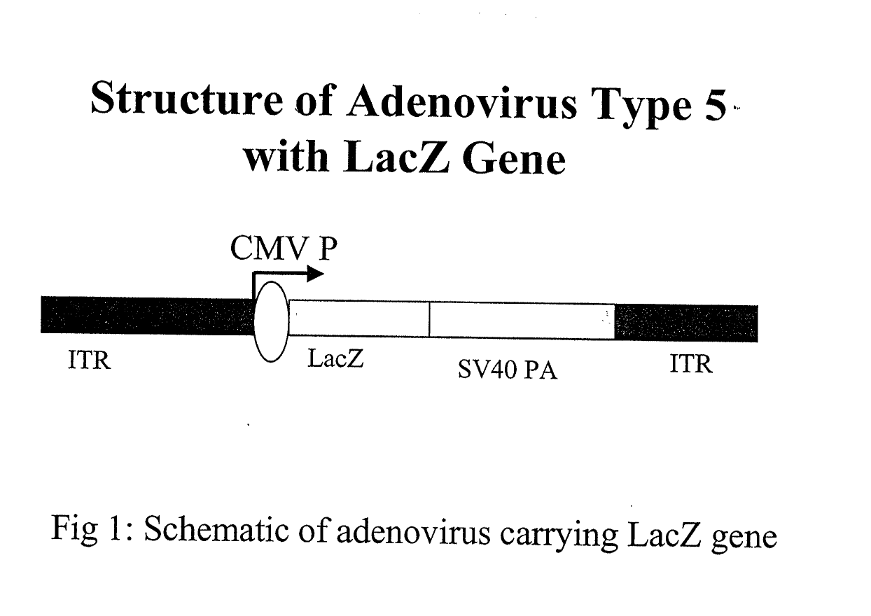

FIG. 2. Effect of Product R on S phase of Hela cell cycle. Cells treatment with Product R and labeled with Propidine Iodide (PI) before or after adenovirus infection. Each bar represents the percentage of S phase DNA content.

1. Hela cells control—Untreated Hela cells.

2. Virus only—Adenovirus control.

3. Virus+Product R—Infection of adenovirus followed by treatment with 10% Product R.

4. Product R/Virus—Cells were first grown in 10% Product R and then infected with adenovirus.

FIG. 3. Effects of Product R on apoptosis of adenovirus infected Hela cells. Infected Hela cells were treated with Product R either contemporaneously or 3 hours later. The cells were labeled with PI 12 hours following infection. The blue bar represents percentage of apoptosis.

1. Hela cells control—Hela cells only;

2. Virus only—Hela cells infected by adenovirus only;

3. Virus+Product R—infection of adenovirus first, 3 hours later the cells were treated by Product R;

4. Product R/Virus—infection of adenovirus and treatment of Product R at same time.

FIG. 4. Ad/CMV/Lac Z expression in Product R treated Hela cells. Hela cells were treated with Product R for 12 hours. Cells were then infected by adenovirus at a concentration of 10 pfu/cell. The LacZ positive cells were counted following day. The blue line represents virus control. The yellow, red, dark line represents blue cells, expression of Ad/CMV/Lac Z in Hela cells.

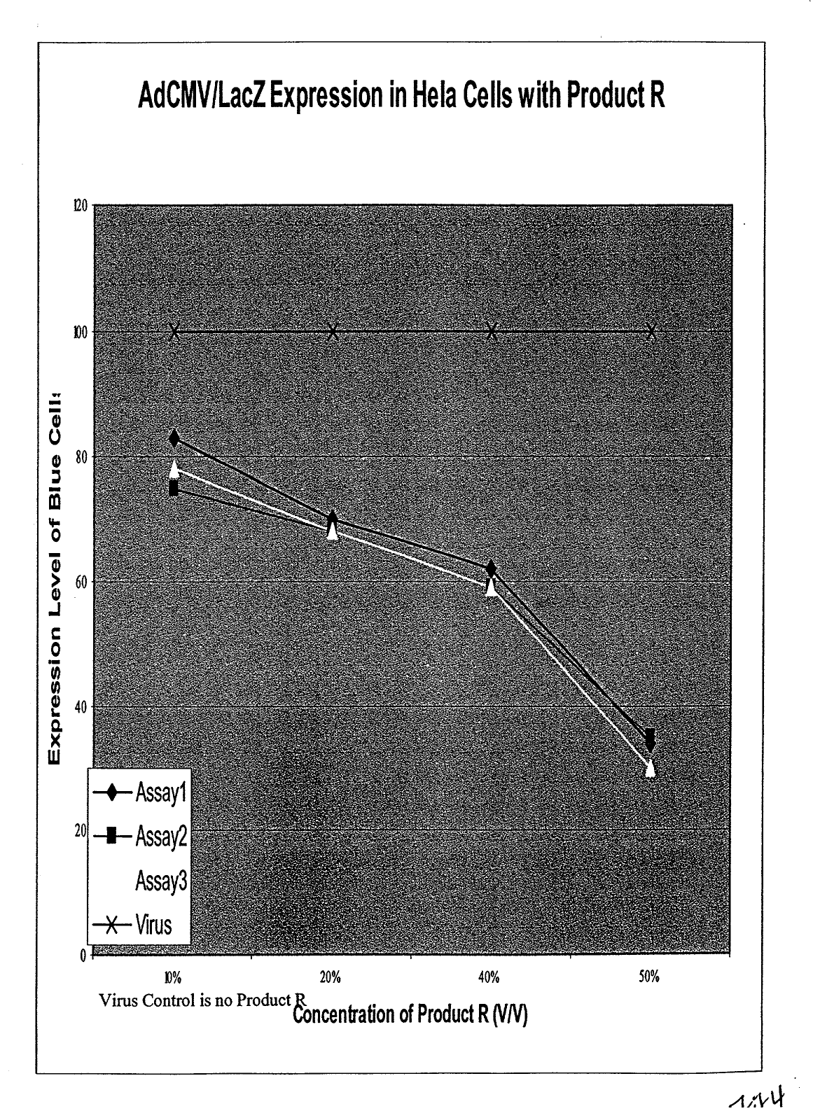

FIG. 5. Inhibition of adenovirus infection in Hela cells. Product R treated Hela cells were assayed for its ability. Yellow line represents virus control; Black line represents adenovirus plaque forming units when treated with interferon- , IFN; Red line represents adenovirus plaque forming units following treatment with Product R.

FIG. 6. Western Blot analysis of E1A protein expression. Hela cells were infected with adenovirus type 5 in the present of 10% Product R. Infected cells were harvested the following day and lysed with NET-N buffer containing protease inhibitors (1 mM PMSF, leupetin [10 ug/ml], aprotinin[1 ug/ml], pepstatin[1 ug/ml], and 1 mM DTT). Equal amounts of protein were separated by SDS-16% PAGE gel, transferred to a PVDF membrane and probed with anti-E1A antibody. The membrane was then striped and probed with an anti-tubulin antibody.

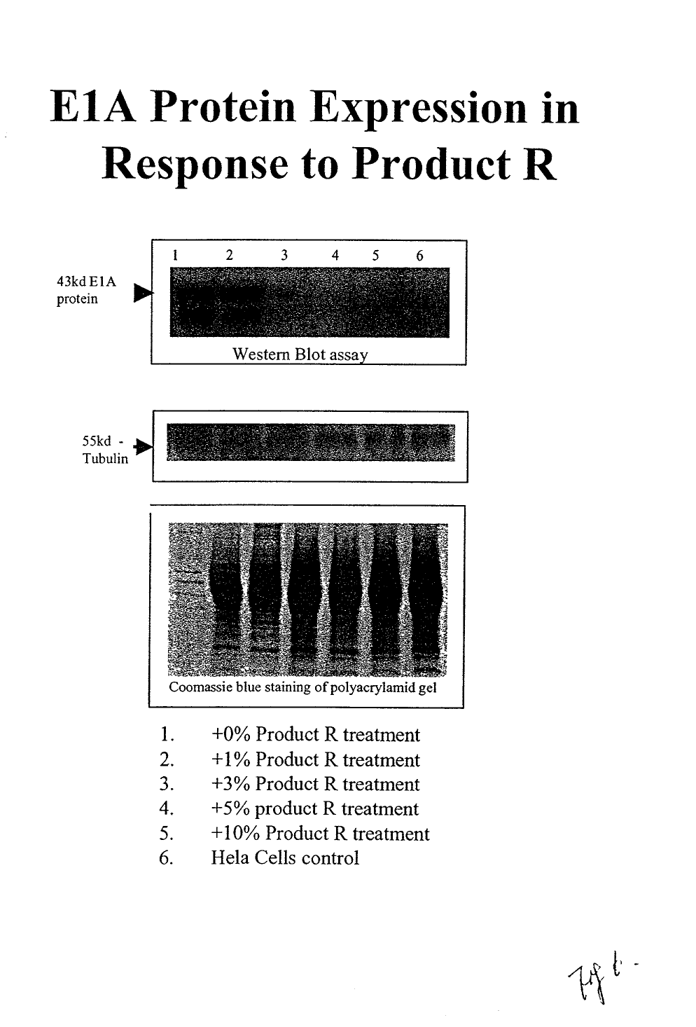

1. The top band represents the 43 Kd E1A protein synthesized in cells treated with varying concentrations of Product R.

2. The middle band represents the 55 Kd human tubulin protein.

3. The bottom bands are sample-loading controls.

FIG. 7. Ad/GFP expression on Hela cells grown in 5% product R. Hela cells were grown for a day in the presence of 5% Product R and infected with 10 pfu/cell of Ad/GFP for 14 hrs. Upper photomicrograph, phase-contrast; Lower, fluorescence. 400×

FIG. 8. Ad/CMV/GFP expression in Hela cells grown in 10% Product R. Hela cells were grown for a day in the presence of 10% Product R and infected with 10 pfu/cell of Ad/GFP for 14 hrs. Upper photomicrograph, phase-contrast; Lower, fluorescence. 400×.

FIG. 9. Ad/GFP expression in Hela cells. Green fluorescence protein marker gene was inserted in the adenovirus EIB region. Hela cells were infected with 10 pfu of Ad/GFP or wild-type adenovirus for 14 hours. Upper phase-contrast; Lower, fluorescence. 400×.

FIG. 10. Ad/CMV/Lac Z expression in Hela cells. Adenovirus carrying Lac Z marker gene was inserted in he adenovirus E1B region. Hela cells were infected with 10 pfu/cell of Ad/CMV/Lac Z virus for 14 hours in 10% Product R treatment. The cells were stained with x-gal, and visualized under microscope at 10×100. The upper photomicrograph represents Hela cells treated with 10% Product R. The lower micrograph shows infected Hela cells grown in the absence of Product R.

### DETAILED DESCRIPTION OF THE PRESENTLY PREFERRED EMBODIMENTS

Product R is manufactured by Advanced Vital Research (Yonkers, N.Y.). The manufacturing process of Product R and its physical and chemical properties redescribed U.S. patent application Serial No. 09/764, 17 filed Jan. 7, 2001.

Other materials described in the following Examples are generally available and can be readily obtained.

The following Examples only serve to illustrate, but not to limit the scope of, the present invention.

### Example 1

### Cells and media

Hela cells were maintained in plates (3×105-4×105 cell/ml) in Dulbecco's modified Eagle's medium supplemented with 10% fetal calf serum and 1 mM sodium pyruvate. Prior to virus infection, Hela cells in plates were grown at 37C.° in the presence of 1 to 10% Product R for 24 hours. Hela cell monolayers were maintained in Dulbecco 's modified Eagle's medium supplemented with 5% fetal calf serum, 5% calf serum, 50 U of penicillin per ml, 50 ug of streptomycin per ml, and 1 mM sodium pyruvate in 5% CO₂ at 37° C.

### Example 2

### Virus infection

Two days before infection, cells were plated at a density of 1.5×10⁴ cells/cm². Various amounts Product R were added to the medium for 14 hours. Stocks of adenovirus-5, laboratory strain Ad/LacZ and Ad/GFP, were used to infect these cells at multiplicity of infection (MOI) of 10 PFU per cell, unless stated differently. Virus adsorption was allowed for 1 hour. Mock-infection control cultures were exposed to an equal volume of medium. At 24 hours post infection, cells were harvested by trypsinization, washed once with cold phosphate-buffer saline (PBS) and aliquots were processed or propidium iodide (PI) binding for flow cytometry. The Ad/GFP and Ad/LacZ viruses were treated as previously described.

### Example 3

### Flow cytometry to assess Product R blocking and E1A expression

A total of 0.5×10⁶ Hela cells were cultured in 10% calf serum and 10% Product R in DMEM medium for 14 hours. Product R treated cells were infected with adenovirus at 10 pfu per cell and incubated for 12 hours. 5×10⁵ cells were suspended in 1 ml FACS buffer. 2ml 100% cold ethanol was added drop wise while vortexing. The cells were kept at −2020 C. for 2 hours. The pellets were incubated with PI solution (50 ug/ml) for 20 minutes and treated with RNase A at 37° C. for 30 minutes. The samples were analyzed by flow cytometry.

### Example 4

### Quantitative PCR analysis to determine viral loads

The number of viral DNA copies per 100,000 cells was determined using the late gene primer pair to estimate the number of proviral sequences and a human GAPGH-specific primer pair was used as an internal standard to assess the number of cell equivalents.

### Example 5

### Western blot analysis

Hela cells were solubilized in Laemmli buffer in the presence of β-mercatpoethanol. Extracts were separated by SDS/PAGE (16% polyacrylamide gel) and electroblotted to a nitrocellulose membrane. Filters were blocked and separately incubated with monoclonal anti-E1A, and with anti-human tubulin monoclonal antibody. Filters were subsequently incubated with a peroxidase-conjugated secondary antibody, and E1A and tubulin were visualized using the Amersham enhanced chemiluminescence system.

### Example 6

### Infectivity inhibition assay

Product R was assayed for its abilities to inhibit adenovirus type 5 infectivity. Product R was diluted to 10% in DMEM-10% FBS, and 50 ul were added in quadruplicate to Hela cells in a 96-well plate for three days until cell to be confluent. After 2 hours incubation at 37° C., 10 pfu of adenovirus were added into Product R-treated and untreated control cells. The virus-Product R mixture was allowed to adsorbed for 2 hours at 37° C., after which the cells were washed and overlaid with DMEM containing 10% FBS and 10% Product R. Five days post-inoculation, the cells were fixed and stained with 1% crystal violet. Plaques were counted, and percent inhibition of virus infectivity in treated wells was determined versus untreated control wells.

### Example 7

### Cell proliferation assay

Cell proliferation assay studies were performed using the Behringer Mannheim cell proliferation Reagent WST-1 (cat# 1644807) method Hela cell were seeded at a concentration of 10×10³ cell/well in 100 ul of culture medium containing various amount of Product R (final concentration: 0, 1%, 5%, 10%) in microtiter plates (tissue culture grade, 96 wells flat bottom). Cells were incubated for 24 -48 hours at 37° C. and 5% CO₂. 10 ul/well Cell Proliferation Reagent WST-1 were added and cells were incubating for 1 hr at 37° C. and 5% CO₂. For background control, 100 ul of culture medium and 10 ul of Cell Proliferation as used in the experiment were added into one well. Background control was used as a blank position for the ELISA reader. Absorbance was measured at 450 nm by a microtiter plate reader and the data were processed using Microsoft Excel.

### Example 8

### Analysis of RNA transcripts

Hela cell were incubated for 3 days in the presence or absence of different concentrations of Product R or 20 U/ml of interferon-α(PBL). Cells infected with adenovirus were incubated in different concentrations of Product R for 3 days. Total RNA was isolated from cells using UltraspEC™—llRNA isolation system (Biotecx, Tex.). For reverse transcriptase (RT-PCR) analysis, 4 ug of total RNA was reversely transcribed into cDNA using adenovirus E 1A and L2 specific oligonucleotides.

Results

Product R did not significant affect cell proliferation of fibroblasts. By proliferation reagent WST-1 assay, fibroblast cells wasn't effected in treatment of Product R. There is slightly inhibition of cell proliferation of Hela cells in 10% Product R. Thus, the fibroblast cells are more tolerance than Hela cell to product.

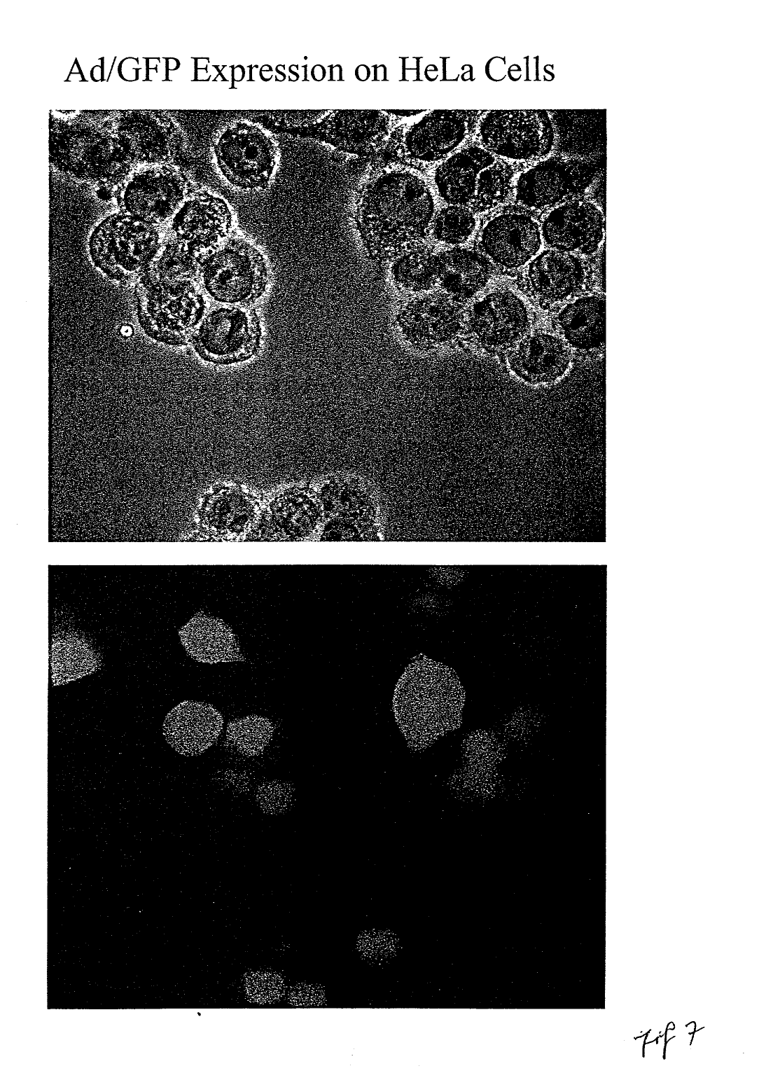

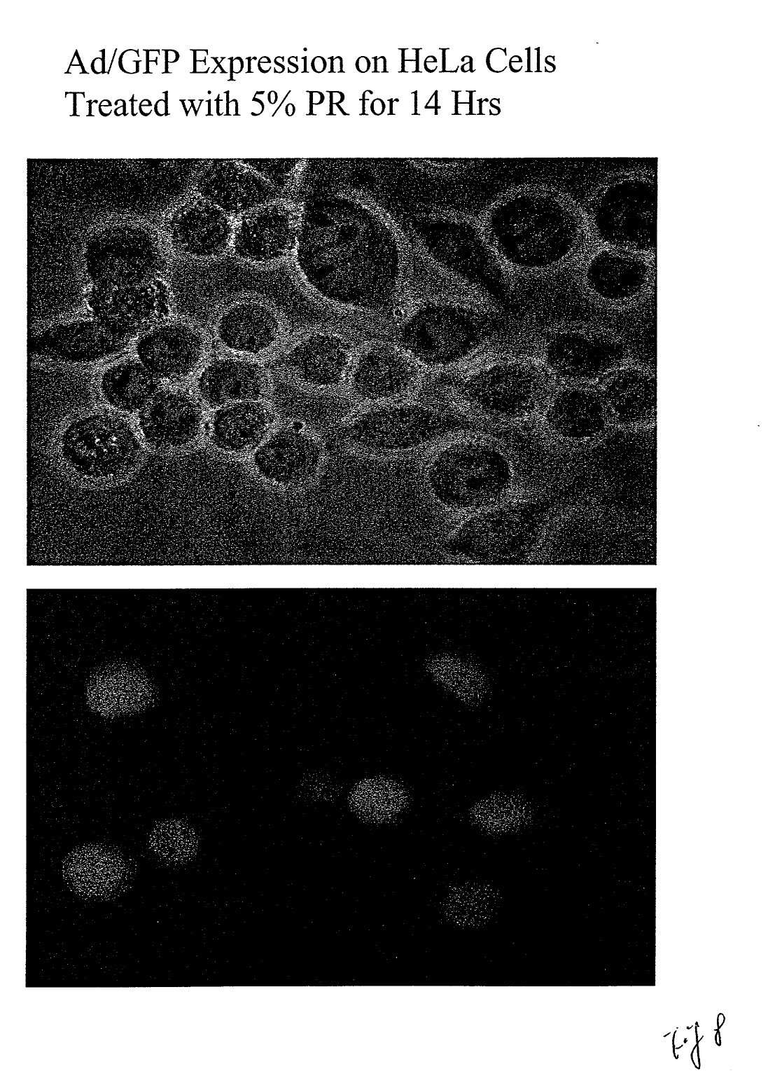

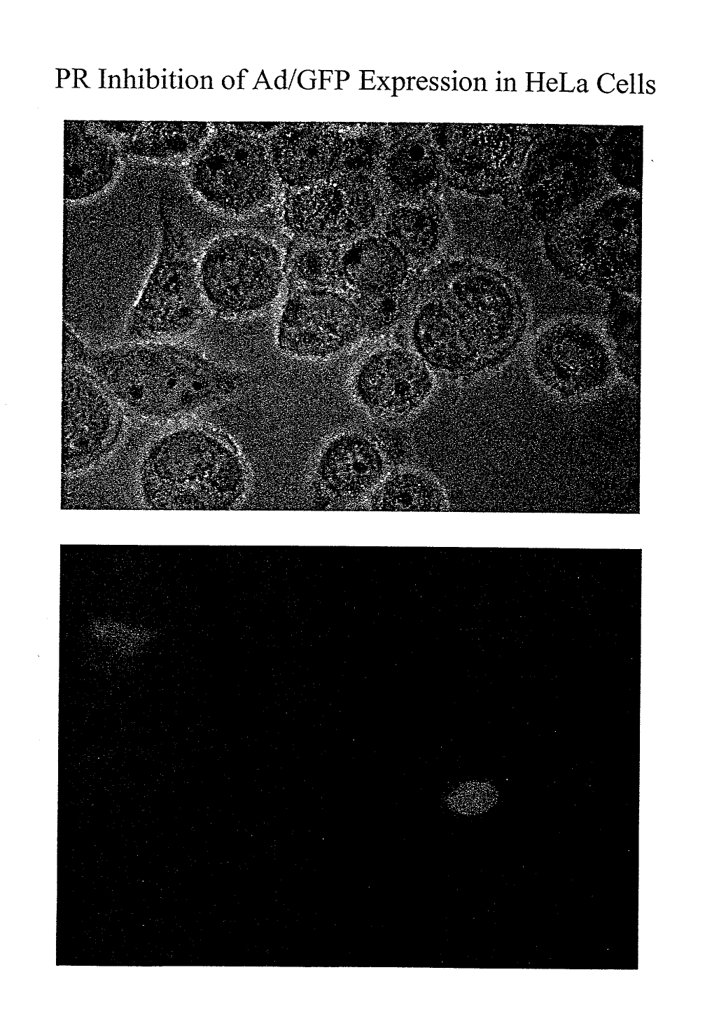

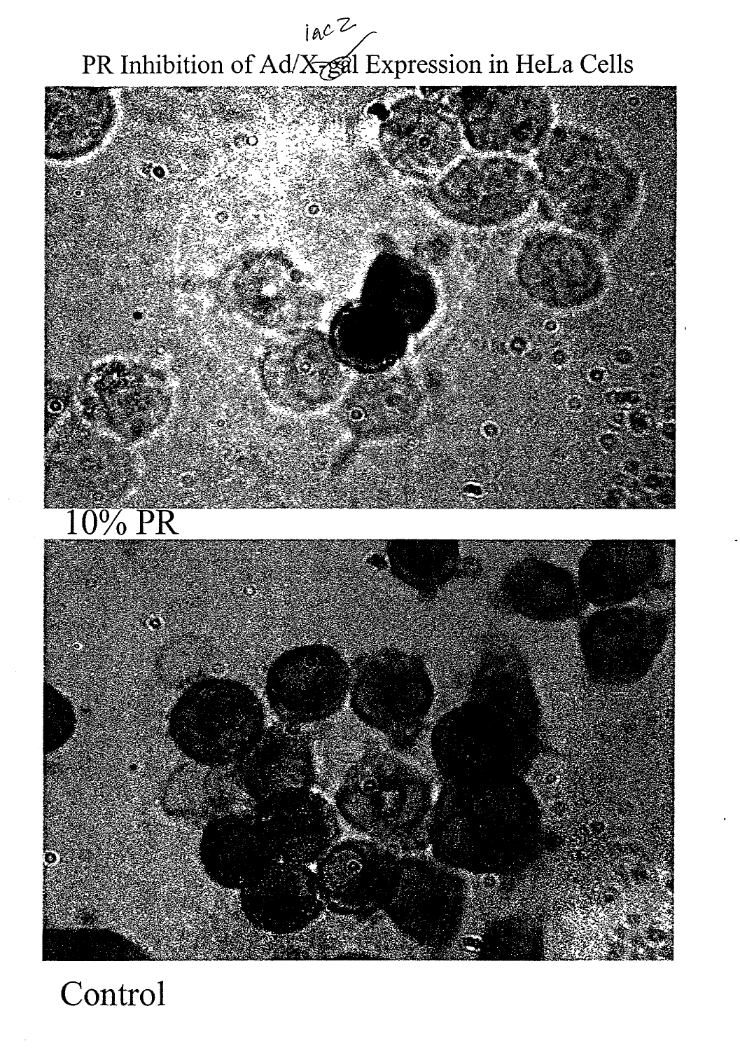

Adenovirus expression was inhibited by Product R. To test the effect of Product R on adenovirus expression, cells were incubated in the presence of 10% Product R and the expression of Adenovirus E1B fused GFP and E1A fused lacZ were assayed by cytoflow directly visualized by fluorescence microscopy. The results demonstrated inhibition of E1A and E1 B fused protein expression in cells treated with Product R, as shown by decreased of green or blue cells as in FIGS. 7, 8, 9 and 10.

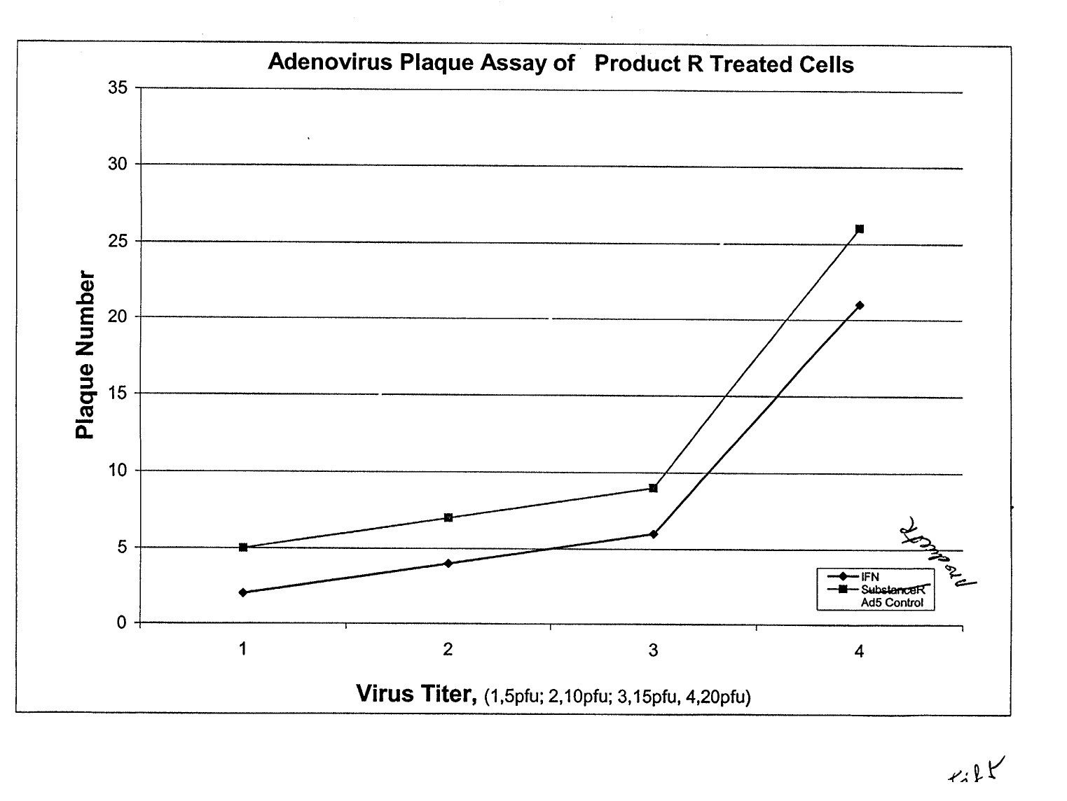

Product R reduced the adenovirus plaque formation. Plaque forming ability is a main characteristic of normal adenovirus particle assembly. Any later gene disruption can cause the abortion of viral plaque formation. Product R inhibited adenovirus plaque numbers in a dose dependent manner. The Product R effect was compared with that of interferon-α(FIG. 5).

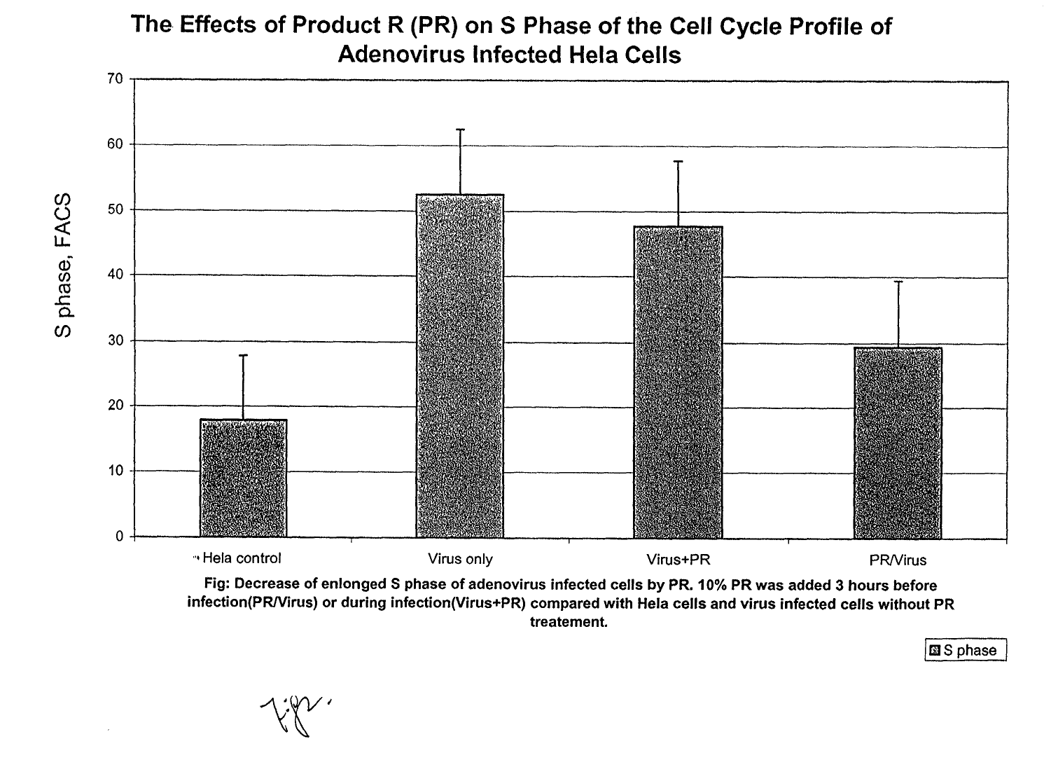

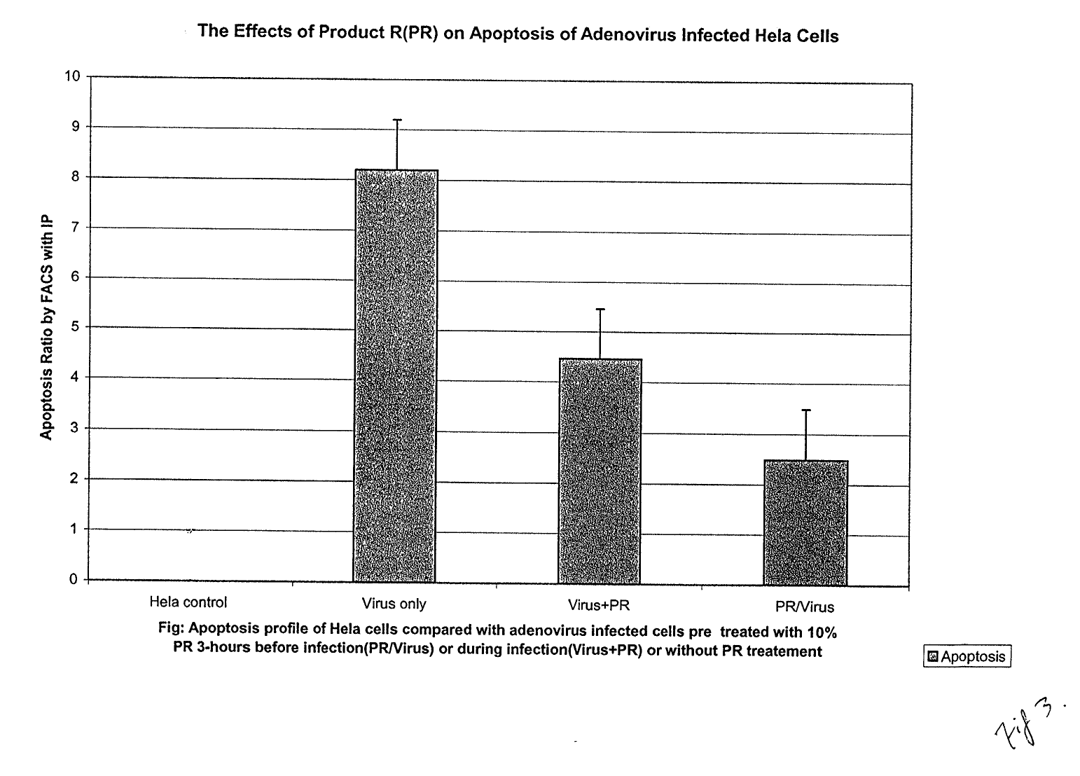

Product R reduced adenovirus-induced apoptosis and modulated the cell cycle. Treatment of adenovirus-infected Hela cell with product R had a marked effect on the position of the cells in the cell cycle. Compared to uninfected and untreated Hela cells, viral infection alone increased the number of cell in S phase and decreased the number in both G1 and G 2, whereas Product R treatment alone decreased the number in S phase and increase the number in G 1. When Product R pre-treated cells were subsequently infected with virus, the cell population resembled that of virally infected cells, except that a larger percentage of cells were in G2. (FIG. 2). In contrast, Product R treatment of virally-infected cells significantly increased the number of cells in both G1 and G 2, while significantly decreasing the number of cells in S phase. Apoptosis was decreased by Product R under these conditions (FIG. 3).

### Conclusions

The results establish that Product R inhibits adenovirus replication as measured by the reduction in both visible viral plaques and the expression on the maker genes LacZ or green fluorescence protein. Product R reduces adenovirus induced apoptosis and modulates the cell cycle. The ability of Product R to alter steady state levels of adenovirus E1A protein suggests that at least these effects are transmitted, to certain extent, via modulation of this viral regulatory gene.

Thus, while there have shown and described and printed out fundamental novel features of the invention as applied to a preferred embodiment thereof, it will be understood that various omissions and substititions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. Moreover, it should be recognized that structures and/or elements and/or method steps shown and/or described in connection with any disclosed form or embodiment of the invention may be incorporated in any other disclosed or described or suggested form or embodiment as a general matter of design choice. It is the intention, therefore, to be limited only as indicated by the scope of the claims appended hereto.

We claim:

1. A method of inhibiting adenoviral replication comprising the step of contacting an effective inhibition amount of Product with cells infected by adenovirus

2. The method of claim 1, wherein said cells infected by adenovirus are Hela cells.

3. The method of claim 1, further comprising the step of measuring adenovirus E1A gene expression in comparison with cells infected by adenovirus but not contacting Product R.

4. The method of claim 3, wherein said E1A gene expression is measured by Western Blot Assay to determine the amount of E1A protein.

5. The method of claim 3, wherein said E1A gene expression is measured by reverse transcriptase-PCR assay to determine the amount of mRNA produced by said E1A gene.

6. A method of modulating cell cycle comprising the step of contacting an effective modulating amount of Product with said cells.

7. The method of claim 6, wherein said cells are Hela cells.

8. The method of claim 6, wherein said cells are infected by adenovirus.
